(19) United States
(12) Patent Application Publication
(10) Pub. No.: US 2013/0224249 A1
(43) Pub. Date: Aug. 29, 2013

(54) RECOMBINANT TRYPANOSOMA CRUZI CELLS USEFUL AS ANTI-CANCER IMMUNE AGENTS

(71) Applicants: Bruno Galvao Filho, Contagem-mg (BR); Ricardo Tostes Gazzinelli, Belo Horizonte-Mg (BR); Caroline Junqueira Giusta, Belo Horizonte-Mg (BR); Santuza Maria Ribeiro Teixeira, Belo Horizonte -Mg (BR)

(72) Inventors: Bruno Galvao Filho, Contagem-mg (BR); Ricardo Tostes Gazzinelli, Belo Horizonte-Mg (BR); Caroline Junqueira Giusta, Belo Horizonte-Mg (BR); Santuza Maria Ribeiro Teixeira, Belo Horizonte -Mg (BR)

(73) Assignee: LUDWIG INSTITUTE FOR CANCER RESEARCH LTD., New York, NY (US)

(21) Appl. No.: 13/820,656

(22) PCT Filed: Sep. 1, 2011
(86) PCT No.: PCT/US11/50143

§ 371 Date: May 17, 2013

(30) Foreign Application Priority Data
Sep. 3, 2010 (BR) 14100003076

(51) Int. Cl. C12N 15/79; A61K 39/00

(52) U.S. Cl. CPC C12N 15/79; A61K 39/0011
USPC 424/269.1; 435/258.1

(57) ABSTRACT

Recombinant, attenuated parasites are transformed or transfected with nucleic acid molecules which provoke an immunostimulatory and protective response in subjects. Preferably, the parasite is Trypanosoma cruzi, especially strain CL-14, and the transforming nucleic acid molecule encodes for a cancer testis antigen, such as NY-ESO-1.

### FIELD OF THE INVENTION

The present invention relates to a recombinant, attenuated strain of Trypanosoma cruzi useful as an immunostimulant, such as a vaccine. It also relates to methods for making this immunostimulatory vector, as well as to the treatment of conditions which require an improved cell mediated immune response. Such conditions include, but are not limited to cancer and conditions caused by intracellular microorganisms, such as toxoplasmosis, malaria, tuberculosis, and so forth.

### BACKGROUND OF THE INVENTION

One of the major challenges in cancer research is the development of materials which induce effective and long-term immune responses against tumors. The identification of the cancer testis antigen (“CTA”) family as potential immune agents was a tremendous step in this direction; however, the field still requires better forms for delivery of agents such as “CTAs” which induce robust, long-term antigen specific T cell responses, especially CD8⁺ lymphocytes.

Attenuated strains of Trypanosoma cruzi have been used as immunogenic agents. See in this regard published U.S. Patent Application No. 2005/0244437; PCT Application PCT/BE83/00006, and Belgian Application No. BE 89253. These references, as well as the art as a whole, however, do not address the use of strains which contain exogenous nucleic acid molecules. In addition, the attenuated strains are not used in the context of any diseases other than the diseases caused by the organism itself. Further, the methods used for attenuating the strains differ from what is described infra.

Very early work showed that live T. cruzi and extracts thereof were useful tumoricidal agents. See, e.g., Roskin, Cancer Res., 6:363-5 (1946); and Hauschlka, et al., Science, 107:600-602 (1948). More recently, it was shown that extracts of epimastigotes of different genetic groups of T. cruzi inhibited the growth of Erlich adenocarcinoma in rats. See, Batmonkh, et al., Bull Exp. Biol. Med., 142(4):470-3 (2006). The mechanism by which this anti-tumor activity occurs is unknown; however, the data presented herein suggest that initial activation of cells from the innate immune system, by compounds inherent to T. cruzi, may contribute to protective immunity that is more general than pathogen specific.

T. cruzi is able to persist in host tissues and induce long term, antigen specific responses. Also, intrinsic to T. cruzi are agonists for Toll-Like Receptors (“TLRs” hereafter), which are useful in induction of highly polarized Th1 responses. Yet further, the parasite replicates in host cell cytoplasma, which leads to direct antigen presentation through endogenous pathways, with consequent induction of antigen specific, CD8⁺ T cells.

T. cruzi has been used as a host organism for receipt of vectors, which include exogenous nucleic acid molecules. See, for example, DaRocha, et al., Parasitol. Res., 92:113-120 (2004); Lima, et al., Parasitol. Res., 77:77-81 (1991), incorporated by reference in its entirety; and Lima et al., Parasitol Res., 81:6-12 (1995).

The “CL-14” strain of T. cruzi is attenuated, and possesses a less infective phenotype than other strains, most probably because of lower expression of “gp82,” the molecule responsible for interaction of the T. cruzi parasite with surface molecules of the host cell and its invasion. See, Ruiz, et al., Biochem. J., 330:505-11 (1998); incorporated by reference. This feature makes CL-14 four times less infective than its parental CL strain. See, Atayde, et al., J. Parasitol., 34:851-860 (2004).

It has been shown that, following inoculation with CL-14, mice develop protection against subsequent challenge with virulent CL, rather than developing disease. Parasitaemia, symptoms of Chagas disease and death from this infection were prevented. See, Lima, et al., Parasitol. Rev., 77:77-81 (1991); and Lima, et al., Parasitol. Rev., 81:6-12 (1995). Further, Pyhrro, et al., Parasitol. Rev., 84:333-7 (1998), have observed that mice immunized with CL-14, followed by challenge with a more virulent strain of CL, produced IgG1, IgG2a, and IgG2b. See, Pyhrro, et al., supra; Soares, et al., Anais da Academia Brasileira de Ciencias, 75(2):167-72 (2003). The combination of low virulence, and high immunogenicity makes CL-14 a prime candidate for immunological studies.

Current research in stimulating immune responses has focused on protein antigens combined with TLR agonists serving as adjuvants. The latter enhance the activation of dendritic cells (“DCs” hereafter), which favors development of a strong T cell response, and Th1 lymphocytes mediated immunity. See, Wille-Reece, et al., Proc. Natl. Acad. Sci. USA, 102:15190-15194 (2005).

It has now been found that attenuated strains of T. cruzi which have low infectivity, when transformed or transfected with nucleic acid molecules encoding one or more cancer testis antigens, elicits strong, human antigen specific T cell response, and protection against cancer. Hence, a feature of the invention is a recombinant Trypanosoma cruzi cell, transformed or transfected with a nucleic acid molecule, which encodes a cancer testis antigen. The resulting recombinant cell may be used to provoke an immune response against the cancer testis antigen in a subject in need thereof.

How these and other features of the invention are accomplished will be seen in the disclosure which follows:.

### DETAILED DESCRIPTION OF PREFERRED EMBODIMENTS

### Example 1

This example was designed to determine if T. cruzi strain CL-14 could be used, safely, as an innoculant.

C57BL/6 and CD8 ^{-/-}mice were injected, intraperitoneally, with either 10⁷ CL-14 parasites, or 5×10⁵ CL “Brenner strain” parasites, each of which were in the metacyclic trypmastigote form. Parasitemy and mortality were compared, and it was observed that CL-14 did not cause parasitemy in either strain.

In a follow up set of experiments, mice which had received the CL-14 inoculation, were challenged with 5×10³ CL Brenner parasites in blood trypomastigote form, 30 days post inoculation with CL-14, to study onset of parasitemy and mortality. It was observed that the animals which received the CL-14 strain controlled the parasitemy.

Hence, it had been established that, in the in vivo model to be used, CL-14 had low virulence and could be used as a whole cell vector.

### Example 2

This example describes the production of three different recombinant CL-14 strains,

Plasmid pROCKneo, described by DaRocha, et al., supra, was used to transfect the host cells. This vector contains the T. cruzi ribosomal promoter, followed by the ribosomal protein TcPβ2 5′ integenic region, which in turn provides a spliced leader addition site for CTA mRNA. The vector is known to facilitate integration of exogenous genetic material into the T. cruzi β-tubulin locus. This plasmid also contains the 3′-UTR plus integenic sequences from gGAPDH I/ii genes, which provide signal for polyadenylation of CTA genes, and trans- splicing signals for “NeoR” which was used as a drug selection marker.

The coding region for NY-ESO-1, described by, e.g., U.S. Pat. No. 5,804,381 (amino acid sequence: SEQ ID NO: 1), incorporated by reference was fused to a 6× His tag at its 5′ end or, downstream, the signal peptide gp63 was cloned, both using well known techniques. Three constructs resulted: one with NY-ESO-1 alone, one with the 6× His tag, and one with the gp63 fusion. This latter construct was designed to facilitate protein secretion.

Epimastigotes of T. cruzi strain CL-14 were transfected with linearized plasmids prior to transfection, following DaRocha, et al., supra. In brief, samples of 10⁸ parasites in the log phase of growth were rinsed with 1×PBS, centrifuged, and resuspended in electroporation buffer. Then 50 μg of plasmic DNA previously digested with NotI, or water as a mock, was added to the 0,2 cm electroporation cuvettes. After that, the parasites were added. The parasites were electroporated at 0.3 KV and 500 μF, with 2 pulses in intervals of 10 seconds, using the Bio-Rad gene pulse (Bio-Rad). The electroporation product was transferred to the LIT growth medium supplemented with 10% SFB. 24 hours after the electroporation, 250 μg/ml of geneticin was added to the medium to select the transgenic parasites. The period of selection lasted from 3 to 6 weeks. Standard Southern blotting showed integration of the NY-ESO-1 coding sequence into the T. cruzi genome.

### Example 3

The production of recombinant NY-ESO-1 by the CL-14 parasites was determined via Western blotting. To elaborate, protein extracts equivalent to 10⁶ epimastigotes per sample were applied and run in a 15% of SDS PAGE gel, transferred to a nitrocellulose membrane and revealed by incubation with either an anti-NY-ESO-1 mAb, or an anti-RGS-His tag mAh and developed with a goat anti-mouse IgG labeled with horseradish peroxidase. To detect the NY-ESO-1 expression by CL-14-NY-ESO-Igp63SP, the supernatant of culture were concentrated 10× prior to Western blot analysis.

Recombinant protein was found from all samples containing parasite transfected with the recombinant NY-ESO-1. The recombinant NY-ESO-1 which had been tagged with gp63 was found in the cytoplasm of cells, which is consistent with the role of the tagging protein. The recombinant protein produced by the other clones was found in the supernatants of cultures with parasites transfected with NY-ESO-1.

In further studies, immunofluorescence revealed that the recombinant protein was expressed at all life stages of the parasite.

### Example 4

The amastigated form of T. cruzi is the replicative, intracellular stage of the parasite. It persists in host tissue for life, and it is through to be critical for eliciting long lasting CD8⁺ T lymphocytes during T. cruzi infection. Hence, studies were carried out to examine expression of recombinant NY-ESO-1, via amastigotes of recombinant CL-14, in human cell lines.

Cell lines SK-MEL-149, and SK-MEL-52 were used. The former does not normally express NY-ESO-1, while the latter does, and was used as a control.

Samples of cell line SK-MEL 149 were infected overnight, with recombinant metacyclic trypomastigotes of CL-14 which expressed NY-ESO-1, using standard methods. The infection protocol used ten parasites per cell, and took place in RPMI medium supplemented with 10% FBS. The infected cells were stained with anti-NY-ESO-1 specific mAbs.

The results indicated that NY-ESO-1 was present in SK-MEL-149 cell lines infected with CL-14 NY-ESO-His⁺ as well as those infected with CL-14 NY-ESO-1 gp63SP. The negative controls, which were SK-149 cells that were either uninfected, or were infected with wild type CL parasite, showed no production of NY-ESO-1. The location of the protein was consistent with prior studies, which means that the SK-MEL-149 cells infected with CL-14 NY-ESO-1 gp63SP, had the protein hoinogenously diffused in cytoplasm.

### Example 5

In a set of follow up experiments, the ability of the recombinant parasites to stimulate CD4⁺ and CD8⁺ T cells in vitro was assessed.

Antigen presenting cells (“APCs”) were isolated from peripheral blood mononuclear cells (“PBMCs”), and were irradiated following standard methods. These cells were then infected at a rate of 30 parasites per APC, and were then co-cultured with either purified, CD4⁺ T cells, or purified CD8⁺ cells, in accordance with Gnjatic, et al., Proc. Natl. Acad. Sci. USA, 99(18):11813-11818 (2002), incorporated by reference. Cellular immune responses were evaluated via IFN-γ ELISPOT, 72 hours after restimulation with autologous EBV-B cells that had been pulsed with a pool of 17 NY-ESO-1 peptides, which overlap the entire length of the full length NY-ESO-1 protein, or had been infected with recombinant Fowlpox virus expressing NY-ESO-1.

The results indicated that the recombinant parasites were able to elicit NY-ESO-1 specific CD4⁺ T lymphocytes. Confirmation of increase in CD8⁺ T cells was via an assay using a tetrameric construct of HLA*0201, which was compatible with the cells, and a peptide corresponding to amino acids 157-165 of NY-ESO-1.

### Example 6

These experiments were designed to determine the ability of the recombinant parasites to elicit NY-ESO-1 specific humoral and cellular response in vivo.

Wild type (C57 BL/6), and knock out mice were used. Each cohort of animals received two, intraperitoneal doses of 10⁷ T. cruzi, in metacyclic form, with 30 days between the doses. Immune responses were determined 21 days after the second immunization, by collecting sera, and measuring anti-NY-ESO-1 IgG via ELISAs and Western blots, using standard protocols, and by assaying spleen cells with or without one of CD4-NY-ESO-1 specific peptide 90-104 of SEQ ID NO: 1, CD8-NY-ESO-1 specific peptide 127-135 of SEQ ID NO: 1, or T. cruzi derived, immunodominant epitope TSKB 18 peptide ANYDFTLV (SEQ ID NO: 2).

The results indicated that all stably transfected parasites elicited high levels of antibodies specific for NY-ESO-1, especially isotype IgG2c, which is normally induced by IFN-γ produced by CD4⁺ Th1 cells.

Given the antibody response described supra, it is not surprising that the spleen cells exhibited a strong, IFN-γ response when they were stimulated, in vitro, by NY-ESO-1 peptides corresponding to amino acids 90-104 and 127-135 of full length NY-ESO-1, which include epitopes known to be recognized by CD4⁺ and CD8⁺ cells.

In parallel experiments, the IgG2c response to NY-ESO-1, and IFN-γ production were compared, in mice immunized with recombinant parasites, and mice which received a recombinant NY-ESO-1 protein associated with TLRs, mixed with alum. The response to the recombinant parasites was significantly greater.

In an alternative set of experiments, the mice were challenged via a subcutaneous injection of 5×10⁶ B16 melanoma cells, which did or did not express NY-ESO-1. Tumor growth was measured twice a week, for 40 days. The results indicated that NY-ESO-1 specific tumor inhibition had been stimulated.

### Example 7

In follow up experiments, C57B1/6 mice were challenged via subcutaneous injection with either 5×10⁴ B16 melanoma cells or 1×10⁶ CT26 colon adenoma cells. BALB/c mice were challenged with 1×10⁶ CMS5a fibrosarcoma cells. In all cases, the cancer cells did or did not express NY-ESO-1.

Five days after mice received the injection of the cancer cells, they began receiving doses of 10⁷ metacyclic forms of wild type CL-14 parasite, or the same number of recombinant CL-14 parasites. Subject animals received a total of 3 doses, five days apart. Tumor growth and survival of the animals were measured for 40 and 90 days respectively.

The results showed that the repeated injections with the trypomastigote metacyclics which expressed NY-ESO-1 delayed tumor growth and prolonged survival for the mice. No parasitemia or signs of T. cruzi infection were observed, even after 90 days post immunization.

### Example 8

These experiments were designed to elucidate the mechanism by which protective immunity was being induced. The CL-14-NY-ESO-1gp63SP constructs were used, in connection with challenge by B16-NY-ESO-1.

Wild type C57BL/6 mice and knockout mice strains MyD88^{-/-}, IL-12^{-/-}, iNOs^{-/-}, and CD8^{-/-} were used in these experiments. 5×10⁶ splenocytes/ml from immunized wild type and knockout mice were restimulated by culturing the splenocytes in with one of 10 μM of NY-ESO-1 peptide 90-104, 127-135, 10 μg of recombinant NY-ESO-1 protein or T. cruzi peptide TSKB20 (ANYKFTLV) (SEQ ID NO: 3). IFN-γ production was measured three days after restimulation via a standard ELISA.

Splenoeyte samples were collected prior to restimulation and stained with anti-CD3 and anti-CD-8 antibodies as well as tetramers containing NY-ESO-peptide 90-104, 127-135 or T. cruzi peptide TSKB20 (ANYKFTLV) and subsequently assayed by flow cytometry.

It was found that, both MyD88^{-/-} and IL-12p40^{-/-} mice which had been challenged and immunized as described, had severely impaired IFN-γ production. There was no decrease in frequency of NY-ESO-1 specific CD8^{±} T cells in the knockout mice.

Upon challenge with B16-NY-ESO-1, the protective immunity observed supra was not present in either of the aforementioned knock out mice, suggesting that TLRs have a critical role in inducing IL-12 and IFN-γ production, that was seen to be elicited via CL-14-NY-ESO-1gp63SP.

It was also observed that β2 microglobulin^{-/-} CD8^{-/-} mice were unable to control tumor growth, suggesting a critical role for CD8⁺ T cells in host resistance. In contrast, mice which were deficient in iNOS were able to control tumor growth.

The totality of these results indicate that efficacy of the recombinant parasite is dependent upon MyD88 and IL-12, with CD4⁺ and CD8⁺ T cells being the major, cellular sources of IFN-γ.

### Example 9

While the parasites were able to confer protective immunity, it is of course noted that it may be desirable to “clear” subjects of the parasites once a tumor or other condition is deemed to be controlled.

Negative selection markers, such as Herpes simplex thymidine kinase (“HSV-1 TK”) are well known. These markers, in effect, induce cells to “commit suicide” in the presence of drugs such as gangcyclovic and acyclovic. The mechanism by which this takes place is well known and need not be repeated here.

The plasmids pROCKNY-ESO-1 His⁺ and pROCKNYESO-1Gp63 were used and, following standard techniques, coding sequences for HSV-1TK were added. Once it was confirmed that the sequence was present, parasites were treated with acyclovic, and a kill rate of 100% was observed, thus suggesting that negative selection markers might be incorporated into the recombinant parasites.

The foregoing disclosure describes various features of the invention, which relates to a recombinant, attenuated or non-infectious cell, such as a parasitic cell, which has been transformed or transfected with a nucleic acid molecule that encodes a protein or a portion of a protein which generates an immune response effective for alleviating a pathological condition. Tlypanosonia cruzi is the preferred parasite used herein, but other species of Trypanosoma, such as T. brucei may be used, as well as, e.g., Leishmania, Plasmodium, Toxoplasma, Entamalba, and so forth. As these parasites are, of course, infectious agents, they must inherently be, or treated to be, non-infectious or so mildly infectious as to not present an inherent danger to the patient being treated. The skilled artisan will be aware of such strains, as exemplified by CL-14.

The nucleic acid molecule used to transform or transfect the parasite may be any which encode a protein or portion of a protein which induces an immunogenic response.

That immunogenic response is preferably one that is directed against a condition from which a patient is suffering. Cancer antigens, such as “cancer testis antigens” are preferred. In addition to NY-ESO-1, a non-exhaustive list of such antigens includes MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-I, SSX-1, SSX-2(HOM-MEL-40), SSX-1, SSX-4, SSX-5, SCP-I or CT-7.

The parasites may be transformed or transfected by standard methods known to the art. The nucleic acid molecule used encoding the immunogenic protein may be used in combination with, e.g., a nucleic acid molecule which encodes a protein that targets the immunogenic protein to a particular part of the parasite, and/or with a selection marker which permits destruction of the parasites after they have ceased to be therapeutically advantageous.

These recombinant, attenuated parasites may be combined with adjuvants, such as ISCOM, QS-21, alum, CpG, and others known to the art, to produce immunogenic compositions which can then be administered to subjects in need of an improved immunogenic response.

Other features of the invention will be clear to the skilled artisan and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1. A recombinant, attenuated Trypanosoma cruzi (T. cruzi) cell, transformed or transfected with a nucleic acid molecule which encodes a cancer testis antigen (CTA).

2. The recombinant T. cruzi cell of claim 1, wherein said CTA is NY-ESO-1 or an immunogenic protein thereof.

3. The recombinant T. cruzi cell of claim 1, wherein said T. cruzi cell is CL-14.

4. The recombinant T. cruzi cells of claim 1, further transformed or transfected with a negative selection marker.

5. Immunogenic composition comprising the recombinant T. cruzi cell of claim 1, and an adjuvant.

6. A method for stimulating an immunological response in a subject in need thereof, comprising administering to said subject an amount of the recombinant T. cruzi cell of claim 1 sufficient to stimulate an immunological response.

7. The method of claim 1, wherein said subject suffers from cancer.

8. The method of claim 6, wherein said subject suffers from an infectious disease.
